(19) United States
(12) Patent Application Publication
(10) Pub. No.: US 2017/0044469 A1
(43) Pub. Date: Feb. 16, 2017

(54) Chomically Indicating Sterile Wipes

(71) Applicant: Georgia Tech Research Corporation, Atlanta, GA (US)

(72) Inventors: William Judson Ready, IV, Atlanta, GA (US); Lucas John Campbell, Franklin, KY (US); Gabrielle Esposito, Bethlehem, PA (US); Emily Ruth Fitzharris, Atlanta, GA (US); Tara Marie Kovacs, Mount Pleasant, SC (US); Matthew Philip Orr, Mableton, GA (US)

(21) Appl. No.: 15/306,315

(22) PCT Filed: Apr. 23, 2015
(86) PCT No.: PCT/US15/27238

§ 371 Date: Oct. 24, 2016

Related U.S. Application Data
(60) Provisional application No. 61/983,485, filed on Apr. 24, 2014.

(51) Int. Cl. C11D 3/40; C11D 17/00; A61B 90/92; A61L 2/16; B08B 1/00; A61B 90/70; C11D 3/48; C11D 17/04

(52) U.S. Cl. CPC C11D 3/40; C11D 3/48; C11D 17/0039; C11D 17/049; A61L 2/16; B08B 1/006; A61B 90/70; A61B 90/92

(57) ABSTRACT

Aspects of the present disclosure generally relate to sanitizing wipe that provides a visual indication that a sufficient amount of abrasive scrubbing has occurred for a given period of time to properly sterilize various medical devices and medical equipment including needless intravenous hub-and-port systems. The sanitizing wipe can change color when used to properly sanitize medical equipment. The sanitizing swab can comprise a plurality of layers of non-woven material (e.g., cotton) in addition to an indicating film disposed between two layers of non-woven material. The indicating film can comprise a polymeric film and a plurality of microencapsulated dyes incorporated into the polymeric film. The microencapsulated dyes can be adapted to burst upon sufficient force being applied thereto, and the bursting of the microencapsulated dyes can cause the sanitizing wipe to undergo a change in visual state (e.g., change color).

### CROSS REFERENCE TO RELATED APPLICATION

This application claims benefit under 35 U.S.C. §119(e) of U.S. Provisional Patent Application Nos. 61/983,485, filed Apr. 24, 2014, entitled “Chromically indicating sterile wipes (CISW),” which is incorporated herein by reference as if set forth herein in its entirety.

### FIELD OF THE INVENTION

The present disclosure relates to medical device safety and, more particularly, to an apparatus and method for providing a visual indication that a sufficient amount of abrasive scrubbing has occurred for a given period of time to properly sterilize various medical devices and medical equipment including needless intravenous hub-and-port systems.

### BACKGROUND

The development of needleless intravenous (“IV”) administrative systems began in the 1990s to protect against needle stick injuries, which proved dangerous due to the increased spread of blood-borne diseases. The IV systems also provided a means for connecting multiple medicines to a patient without additional needles, thus aiding healthcare professionals and easing patient discomfort.

Unfortunately, these IV systems lead to an estimated 250,000 IV device-related bloodstream infections (“BSIs”) each year in the United States. Each case has an attributable mortality rate of between 12 and 25 percent. It is estimated that these preventable infections make up 4% of medical malpractice claims on doctors and cause hospitals to lose an estimated $9B annually because of nonreimbursed litigation costs.

A majority of BSIs result from bacteria inside hospitals finding its way into patients' bloodstreams, particularly through needleless IV hub-and-port systems. Contamination of the hub-and-port system often occurs when the port hub is exposed to environmental elements during IV changeover, thus resulting in an unsanitary port connection.

The so-called Scrub-the-Hub Protocol (the “Protocol”) establishes an approach to ensure that hubs, ports, and connectors are properly cleaned to eliminate the risk of BSIs. Typically, alcohol- or chlorhexidine-soaked swabs are used to abrasively scrub the components of an IV system for a given period of time to remove viruses, bacteria, yeast, fungi, and other biofilms that can cause BSIs. Because of the ease with which they can be prevented using the Protocol, the Center for Medicare and Medicaid characterizes BSIs as “never events” (i.e., events that should never occur).

Unfortunately, studies question compliance with the Scrub-the-Hub Protocol. In particular, research suggests that IV systems often are not scrubbed for the recommended duration or with the proper amount of friction to remove biofilms. For example, the Protocol recommends cleaning IV ports for at least 15 seconds and some facilities recommend cleaning for 30 seconds, but current estimates reveal that most healthcare professionals clean the IV ports for approximately nine seconds.

Accordingly, there is a need for improved systems and methods to address the above mentioned deficiencies. Embodiments of the present disclosure are directed to these and other considerations.

### SUMMARY

Briefly described, and according to one embodiment, aspects of the present disclosure generally relate to an apparatus and method for providing a visual indication that a sufficient amount of abrasive scrubbing has occurred for a given period of time to properly sterilize various medical devices and medical equipment including needless intravenous hub-and-port systems. The apparatus may be embodied as a sanitizing swab, which may alternatively be referred to as a “wipe” or a “sanitizing wipe.” The sanitizing swab may comprise a plurality of layers of non-woven material or fabric (e.g., cotton). The sanitizing swab may further comprise an indicating film disposed between a first and second layer of non-woven material, and the indicating film may comprise a polymeric film and a plurality of microencapsulated dyes incorporated into the polymeric film. Further, the plurality of microencapsulated dyes burst upon sufficient uniaxial force being applied thereto and the bursting of the plurality of microencapsulated dyes causes the sanitizing swab to undergo a change in visual state.

In one embodiment, a method for producing a sanitizing swab is provided. The method can include producing, via a spray-drying rotating disk process, a plurality of microencapsulated dyes. Further, the method may include incorporating the plurality of microencapsulated dyes into a polymeric film to generate an indicating film. The method can also include disposing, using a laminating process, the indicating film between a first layer of non-woven material and a second layer of non-woven material.

These and other aspects, features, and benefits of the present disclosure will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings, which are incorporated in and constitute a part of this specification, illustrate multiple embodiments of the presently disclosed subject matter and, together with the description, serve to explain the principles of the presently disclosed subject matter; and, furthermore, are not intended in any manner to limit the scope of the presently disclosed subject matter. Wherever possible, the same reference numbers are used throughout the drawings to refer to the same or like elements of an embodiment, and wherein:

FIG. 1 is an overview 100 of a healthcare practitioner cleaning an exemplary connector used in an IV system.

FIG. 2A is a view of a chromically indicating sterile wipe (CISW) prior to use, according to an example embodiment of the present disclosure.

FIG. 2B is a view of a CISW after use according to a prescribed protocol, according to an example embodiment of the present disclosure.

FIG. 3 is an exploded view of a CISW, according to an example embodiment of the present disclosure.

FIG. 4 is a view of a system for manufacturing chromically indicating sterile wipes, according to an example embodiment of the present disclosure.

FIG. 5 is a flow chart of a process for manufacturing a CISW, according to an example embodiment of the present disclosure.

FIG. 6A is a view of a CISW prior to use, according to an example embodiment of the present disclosure.

FIG. 6B is a view of a CISW after use according to a prescribed protocol, according to an example embodiment of the present disclosure.

### DETAILED DESCRIPTION

Certain embodiments of the disclosed technology provide an apparatus and method for properly cleaning medical equipment according to prescribed guidelines. In particular, certain embodiments provide an apparatus and method for providing a visual indication that a sufficient amount of abrasive scrubbing has occurred for a given period of time to properly sterilize various medical devices and medical equipment including needless intravenous hub-and-port systems.

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an” and “the” include plural references unless the context clearly dictates otherwise. References to a composition containing “a” constituent is intended to include other constituents in addition to the one named. Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

The materials described as making up the various elements of the present invention are intended to be illustrative and not restrictive. Many suitable materials that would perform the same or a similar function as the materials described herein are intended to be embraced within the scope of the present invention. Such other materials not described herein can include, but are not limited to, for example, materials that are developed after the time of the development of the present invention.

Example embodiments of the disclosed technology will now be described with reference to the accompanying figures.

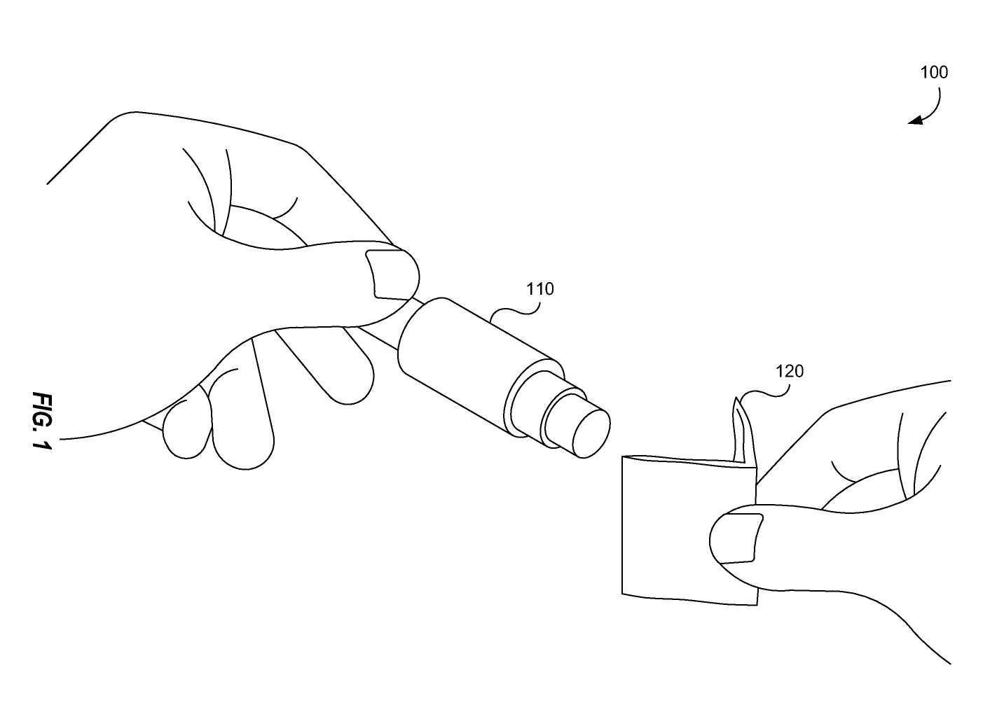

As discussed, intravenous (“IV”) systems commonly are used in healthcare for delivering drugs, blood, and nutrients quickly into the body. FIG. 1 is an overview 100 of a healthcare practitioner cleaning an exemplary IV connector 110 of a conventional IV system. Generally, a typical IV system comprises an IV port, which can be placed under a patient's skin, and an IV connector 110 such as shown in FIG. 1, which can be used for delivering drugs, blood, and nutrients into the port from, for example, and external bag.

As will be appreciated, proper cleaning of both ports and connectors is imperative to maintain a sterile state. In particular, hospitals and other entities such as the Centers for Disease Control (“CDC”) establish protocols to ensure that the ports and connectors are properly cleaned to reduce the risk of device-related bloodstream infections (“BSIs”) or central-line associated bloodstream infections (“CLABSIs”). The CDC established The CDC established the Hemodialysis Central Venous Catheter Scrub-the-Hub Protocol (the “Scrub-the-Hub Protocol” or the “Protocol”) that mandates that IV ports and connectors be cleaned prior to use. Generally, as shown in FIG. 1, healthcare practitioners use an alcohol swab 120 to scrub IV system components such as the IV connector 110 to remove viruses, bacteria, and fungi that can cause BSIs.

Recent studies, however, question whether medical practitioners follow the sterilization protocols and, in particular, the Scrub-the-Hub Protocol. Specifically, studies show that practitioners do not scrub IV system components for the recommended duration or with the proper amount of friction to properly remove biofilms that promote bacterial growth.

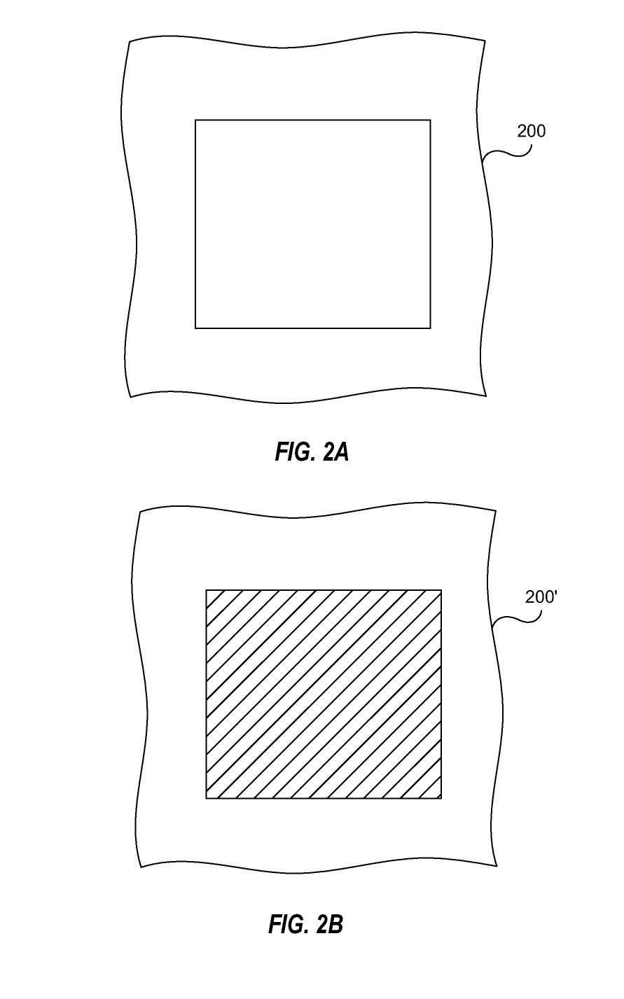

Aspects of the present disclosure relate to chromically indicated sterile wipes (“CISWs”), such as CISW 200 shown in FIG. 2A, that provide a visual indicator for the practitioner when the swabs are used according to established protocols. For example, in some embodiments, when a practitioner uses a CISW 200 according to an established protocol (e.g., for a predetermined period of time, with a proper amount of pressure, etc.), as shown in FIG. 2B, the color of the CISW 200′ will change distinctly. Thus, in some embodiments, a CISW 200 can be both time-dependent and pressure-sensitive.

In some embodiments, a CISW 200 can comprise a pressure and/or friction responsive microencapsulated dye that can be time-activated and that can rupture upon successful use of the CISW 200 according to a prescribed protocol. So, for example, in some embodiments, a CISW 200 can comprise a microencapsulated dye that ruptures after between 5 and 30 seconds of vigorous rubbing, thus resulting in a change of color to the CISW 200′, as shown in FIG. 2B.

Further, in some embodiments, a CISW 200 can comprise a microencapsulated dye that ruptures when the practitioner scrubs the IV component vigorously or abrasively according to a prescribed protocol. So, for example, in some embodiments, a CISW 200 can comprise a microencapsulated dye that ruptures when a practitioner uses the CISW 200 to scrub an IV component with a predetermined force as low as approximately 0.5 and up to approximately 30 PSI (or between approximately 3,400 and 207,000 Pa) depending on the particular application, protocol, and need. As will be appreciated, 2 PSI is approximately twice the adhesion force of an E. coli bacterium and thus may be adequate to sanitize surfaces, though some protocols or applications may require greater pressure.

As will be understood by one of skill in the art, a microcapsule generally is a capsule having a coating that surrounds particles or droplets. Put differently, a microcapsule is a small sphere with a uniform wall around it and a material inside. According to some embodiments of the present disclosure, a microencapsulated dye is a microcapsule comprising a dye or other antiseptic.

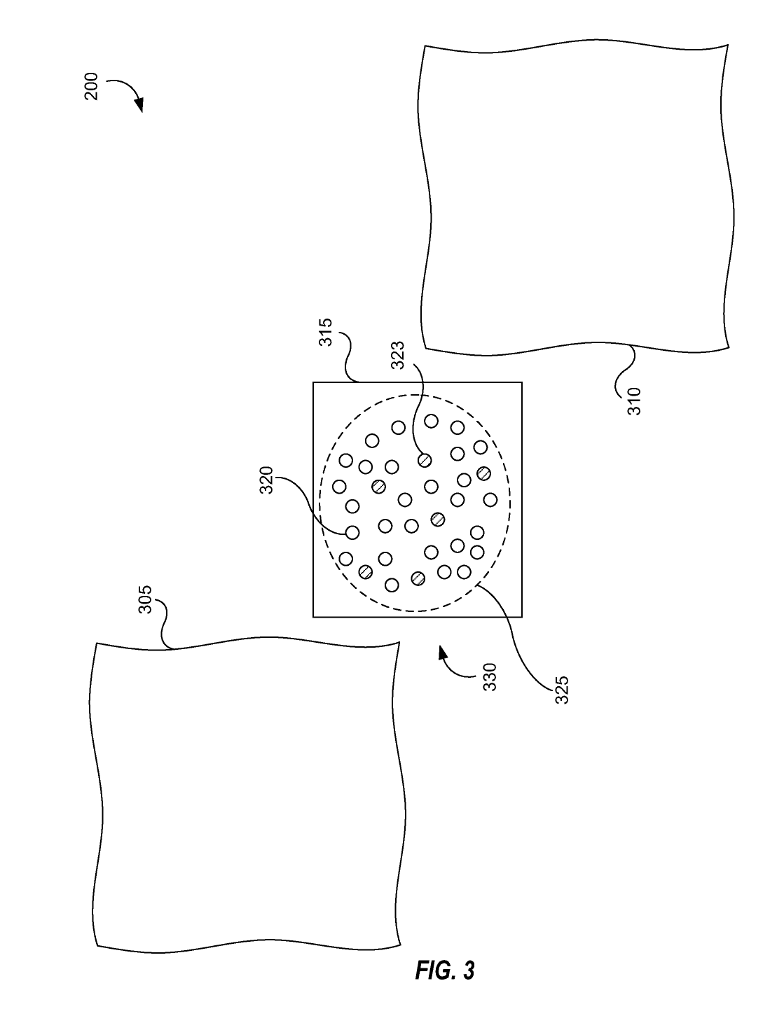

FIG. 3 is an exploded view of a CISW 200, according to some embodiments of the present disclosure. In some embodiments, a CISW 200 according to the present disclosure has the look and feel of well-known “wet wipes” gauze products. As discussed, in some embodiments, a CISW 200 can comprise microencapsulated dyes that rupture through a combined time- and pressure-dependency to indicate compliance with a prescribed protocol. As shown in FIG. 3, in some embodiments, a CISW 200 can comprise a first substrate 305 and a second substrate 310. For example, in some embodiments, the first and second substrates 305, 310 can be cotton gauze plies or other non-woven material.

Further, in some embodiments, a CISW 200 can comprise a plurality of microencapsulated dyes, shown in FIG. 3 individually as microencapsulated dye 320 and collectively by dashed line 325. As discussed, in some embodiments, microencapsulated dyes 325 can be configured to rupture following a time-dependent abrasive/fatigue failure mode process in which the microencapsulated dyes 325 rupture after a CISW 200 has been used according to a prescribed protocol (and not rupture, for example, if the microencapsulated dyes 325 are compacted suddenly (e.g., as can occur during a lamination process or during shipping and/or storage). As will be understood, when proper pressure is applied for a predetermined duration according to the prescribed protocol, and thus sufficient force is applied to the microencapsulated dyes 325, the microencapsulated dyes 325 burst, and the dye provides a visual indication to the practitioner that the IV component has been sanitized according to the protocol. In some embodiments, the microencapsulated dyes 325 burst and react with a receiver paper incorporated into the CISW 200 such that the receiver paper undergoes a change in visual states.

As will be appreciated, in to a CISW 200 meeting requisite FDA cleanliness and sterilization guidelines, when used in a medical setting, microencapsulated dyes 325 included in a CISW 200 should be nontoxic. For example, in some embodiments, the dyes can be water- or alcohol-based. Further, in some embodiments, in addition to microencapsulated dyes, a CISW 200 may include microcapsules 323 (e.g., pressure-sensitive microcapsules) that encapsulate an antiseptic (e.g., iodine or isopropyl alcohol). As will be appreciated, these iodine- or alcohol-filled microcapsules 323 can further enhance the antiseptic qualities of a CISW 200.

In some embodiments, microencapsulated dyes 325 can be incorporated within a polymeric film 315. As will be appreciated, incorporating the microencapsulated dyes 325 into a polymeric film 315 can help prevent dye from coming into contact with a patient or IV system component. In some embodiments, the polymeric film 315 and incorporated microencapsulated dyes 325 can form an indicating film 330 (alternatively, a reactive square), which can be sandwiched between the first and second substrates 305, 310 to form a CISW 200.

In some embodiments, microencapsulated dyes 325 can be incorporated into a CISW 200 through dispersion. For example, the microencapsulated dyes 325 can be dispersed in an isopropyl alcohol solution that is subsequently absorbed into individual layers of non-woven fibers (e.g., first substrate 305 and second substrate 310). The individual layers (e.g., 305, 310) can then be pressed together to form a CISW 200.

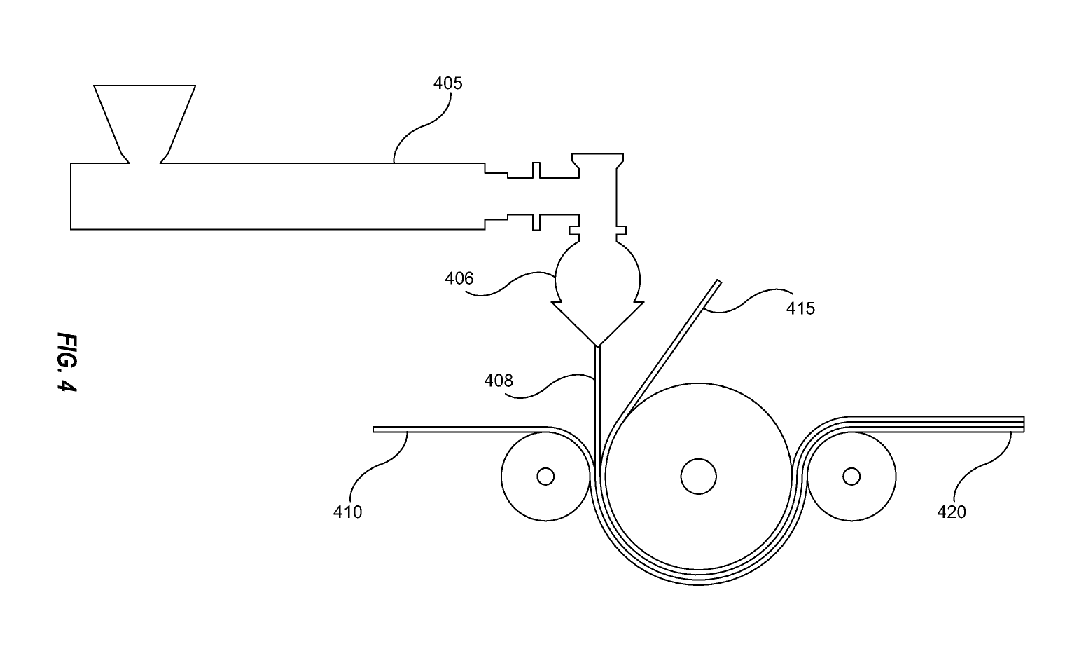

As discussed, in some embodiments, a CISW 200 can comprise an indicating film 330 comprising a polymeric film 315 and incorporated microencapsulated dyes 325. As shown in FIG. 4, in some embodiments, an indicating film 408 can be laminated as an inner layer between a first substrate 410 and a second substrate 415, both of which can be nonwoven gauze plies. Subsequently, in some embodiments, the indicating film 408 and the first and second substrates 410, 415 can be infiltrated with an antiseptic (e.g., alcohol) and/or an antibacterial (e.g., chlorhexidine) using a roll-to-toll manufacture process. In some embodiments, microencapsulated dyes (e.g., 325) can be produced in a spray-drying rotating disk process in an extruder 405 and then be incorporated into a polymeric film or low-density polymeric film (e.g., 315) to form an indicating film 408 that is extruded from an extrusion die 406. As will be understood by one of skill in the art, spray drying is a method for microencapsulation in which an active material (e.g., dye, antiseptic) is suspended or dissolved in a polymer solution such that the active material becomes trapped inside the dried particle. In some embodiments, after extrusion, the indicating film 408 is sandwiched between the first and second substrates 410, 415 to yield an extrusion laminate 420 that can be cut into individual CISWs 200. For example, an individual CISW 200 can be approximately 9 square inches.

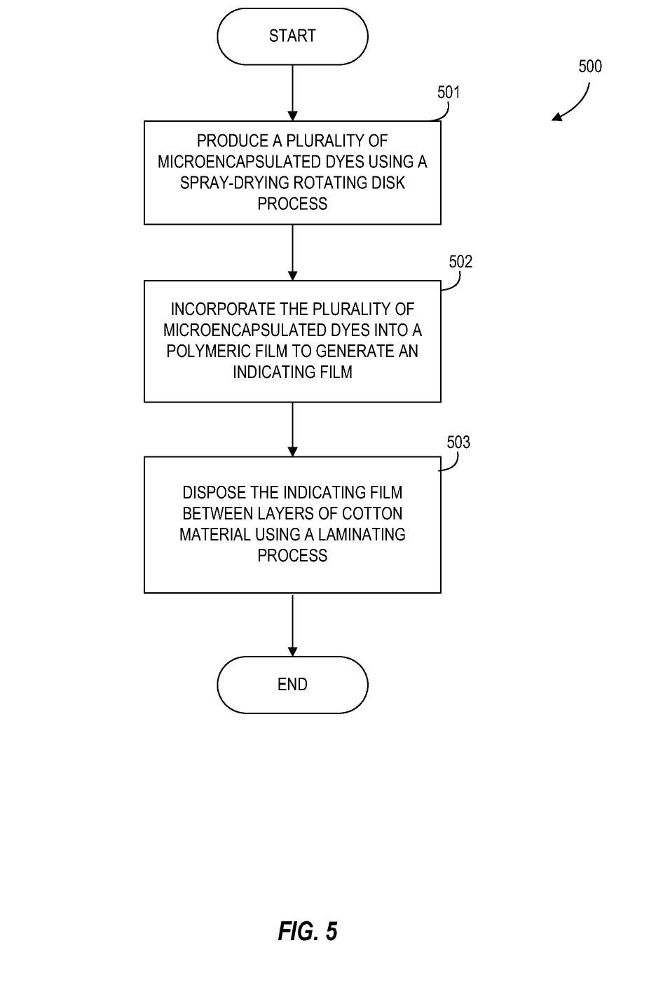

FIG. 5 is a flow chart of a process 500 for manufacturing a CISW 200, according to some embodiments. As shown in FIG. 5, in some embodiments, a process 500 for manufacturing a CISW 200 can comprise producing a plurality of microencapsulated dyes (e.g., 325) using a spray-drying rotating disk process, at 501. Further, at 502, the process 500 can comprise incorporating the plurality of microencapsulated dyes 325 into a polymeric film (e.g., 315) to generate an indicating film (e.g., 330). Finally, in some embodiments, the process 500 can comprise disposing the indicating film 330 between layers of non-woven material (e.g., 305, 310) using a laminating process, at 503.

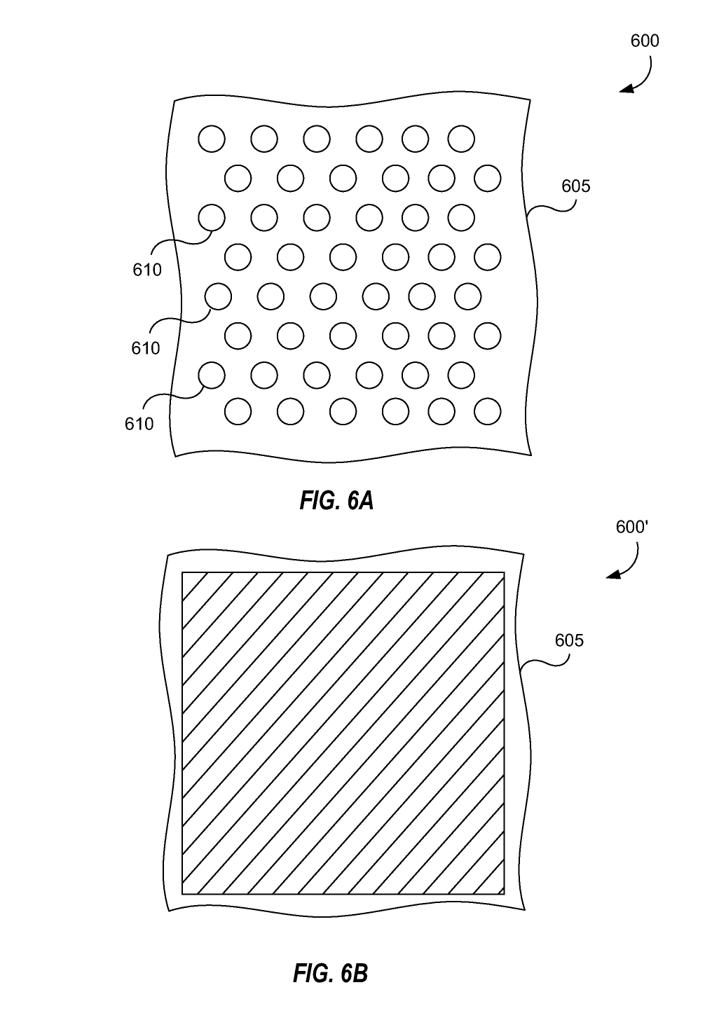

As shown in FIG. 6A, in some embodiments, a CISW 600 can comprise a plurality of microencapsulated dyes 610 that are distributed approximately uniformly throughout the non-woven material 605 comprising the CISW 600. In such embodiments, the plurality of microencapsulated dyes 610 can be dispersed in, for example, chlorhexidine and infiltrated (i.e., distributed) throughout the non-woven material 605. Accordingly, when a CISW 600 according to this embodiment is used to clean an IV component, rubbing pressure can cause the plurality of microencapsulated dyes 610 to burst (i.e., rupture), which allows the dye to leak into the non-woven material 605. Additionally, in some embodiments, when the microencapsulated dyes 610 burst, the dye also can leak onto the IV component being sanitized. In some embodiments, the encapsulated dye is transient and thus fades from the surface of the IV component after a period of time. FIG. 6B illustrates a CISW 600′ after the microencapsulated dyes 610 burst and the dye leaks onto the non-woven material 605.

In some embodiments, a CISW (e.g., CISW 200) may comprise microcapsules containing thermochromic dyes (i.e., leuco dyes) that are incorporated into the non-woven material. As will be understood by one of skill in the art, leuco dyes generally comprise molecules that have two forms, one colorless and the other colored, that change based on temperature. Thus, in some embodiments, heat from a practitioner's hands and/or heat created by rubbing a CISW on an IV component can cause a rise in temperature, which in turn causes a color change, which is reversible. To maintain the reversibility, the leuco dye typically is encapsulated in a protective polymer shell. In some embodiments, thermochromic liquid crystals (“TLCs”) can be substituted for leuco dyes. Typically, the TLCs undergo changes in pitch with temperature change, thus altering the Bragg's reflection of light that emits from the material. Further, in some embodiments, using multiple TLCs in a CISW can increase the range of temperature in which the CISW retains functionality, thereby increasing the range of colors achieved while the CISW is in use.

While certain embodiments of the disclosed technology have been described in connection with what is presently considered to be the most practical and various embodiments, it is to be understood that the disclosed technology is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the disclosed technology, including the best mode, and also to enable any person of ordinary skill to practice certain embodiments of the disclosed technology, including making and using any devices or systems and performing any incorporated methods. The patentable scope of certain embodiments of the disclosed technology is defined in the claims, and may include other examples that occur to those of ordinary skill. Such other examples are intended to be within the scope of the claims if they have structural elements that do not differ from the literal language of the claims, or if they include equivalent structural elements with insubstantial differences from the literal language of the claims.

1. A sanitizing swab comprising:
layers of non-woven material; and
an indicating film disposed between a first layer of non-woven material and a second layer of non-woven material, the indicating film comprising:
a polymeric film; and
microencapsulated dye incorporated into the polymeric film;
wherein at least a portion of the microencapsulated dye releases upon an activation event; and
wherein the release of dye causes the sanitizing swab to undergo a change in visual state.

2. The sanitizing swab of claim 1, wherein the activation event is selected from the group consisting of releasing dye from at least a portion of the microencapsulated dye upon a sufficient force and releasing dye from at least a portion of the microencapsulated dye upon a sufficient period of time; and
wherein the change in visual state indicates that the sanitizing swab has been used according to a prescribed sanitizing protocol.

3. The sanitizing swab of claim 2, wherein the prescribed sanitizing protocol is Scrub-the-Hub Protocol.

4. The sanitizing swab of claim 2, the indicating film further comprising microencapsulated antiseptic.

5. The sanitizing swab of claim 4, wherein the antiseptic is selected from the group consisting of iodine, chlorhexidine, and isopropyl alcohol.

6. The sanitizing swab of claim 2 further comprising a receiver paper disposed between the first layer of non-woven material and the second layer of non-woven material, wherein, upon the activation event, dye reacts with the receiver paper such that the receiver paper undergoes the change in visual state.

7. The sanitizing swab of claim 2, wherein the dye is a water-based dye.

8. (canceled)

9. The sanitizing swab of claim 2, wherein the sufficient period of time is between approximately 5 and 30 seconds.

10. The sanitizing swab of claim 2, wherein the sufficient force is sufficient to remove biofilms from a surface.

11. The sanitizing swab of claim 2, wherein the sufficient force is between approximately 0.5 and 30 PSI.

12. A method of producing the sanitizing swab of claim 1 comprising:
producing, via a spray-drying rotating disk process, the microencapsulated dye;
incorporating the microencapsulated dye into the polymeric film forming the indicating film; and
disposing, using a laminating process, the indicating film between the first layer of non-woven material and the second layer of non-woven material.

13. The method of claim 12 further comprising:
infiltrating the indicating film, the first layer of non-woven material, and the second layer of non-woven material with at least one of an antiseptic and an antibacterial.

14. The method of claim 12 further comprising:
activating the activating event to release at least a portion of the microencapsulated dye.

15.-16. (canceled)

17. The method of claim 14, wherein the activating event comprises a force sufficient to remove biofilms from a surface.

18. The method of claim 14, wherein the activating event comprises a period of time.

19. (canceled)

20. A sanitizing swab comprising:
a layer of non-woven material; and
microencapsulated dye that is distributed approximately uniformly throughout the layer of non-woven material;
wherein at least a portion of the microencapsulated dye bursts upon a sufficient force being applied thereto; and
wherein dye released from the at least a portion of burst microencapsulated dye is adapted to leak onto the layer of non-woven material to cause the sanitizing swab to undergo a change in visual state; and

[symbol]

21. The sanitizing swab of claim 20, wherein the microencapsulated dye is transient.
